(19) United States
(12) Patent Application Publication
(10) Pub. No.: US 2008/0076131 A1
(43) Pub. Date: Mar. 27, 2008

(54) Methods and devices for nucleic acid amplification on a surface

(76) Inventors: Alexander M. Chagovetz, Salt Lake City, UT (US); Steven M. Blair, Salt Lake City, UT (US)

Correspondence Address: THORPE NORTH & WESTERN, LLP., P.O. Box 1219, SANDY, UT, 84091-1219, US

(21) Appl. No.: 11/809,726

(22) Filed: May 31, 2007

Related U.S. Application Data
(63) Continuation-in-part of application No. 11/633,981, filed on Dec. 4, 2006, now abandoned.
(60) Provisional application No. 60/741,688, filed on Dec. 2, 2005.

(51) Int. Cl. C12P 19/34; C07H 21/00; C12Q 1/68; C12M 1/00

(52) U.S. Cl. 435/6; 435/287.2; 435/91.1; 536/25.3

(57) ABSTRACT

Methods of building surface amplification devices are disclosed. Methods and devices for detecting target nucleic acids are also disclosed. Primer pairs are seeded on the surface of a substrate using a connecting compound between the primers to optimize the distance between immobilized primers. The flexible linking compounds avoid the need to bend extension products during later amplifications.

### CROSS-REFERENCE TO RELATED APPLICATION

This application is a continuation-in-part of U.S. patent application Ser. No. 11/633,981 filed on Dec. 4, 2006, which claims the benefit of the filing date of U.S. Provisional Patent Application 60/741,688 filed on Dec. 2, 2005, the contents of the entirety of both of which are incorporated by this reference.

### TECHNICAL FIELD

The invention generally relates to biotechnology, and, more specifically, to the field of diagnostics, such as nucleic acid amplification on a surface.

### BACKGROUND

Nucleic acid amplification in solution-based reactions, either through thermal cycling (e.g., polymerase chain reaction “PCR” and its modifications) or isothermal amplification (e.g., rolling circle amplification “RCA,” Ionian method, Invader) is well established and has been widely used for the last 25 years. There is, however, an intrinsic limitation in solution-based reactions with respect to multiplexing, due to multiple competitive processes, which introduce bias in quantitative features (concentrations) of multiple targets. Two approaches emerged to overcome these limitations: non-specific whole genome amplification through the use of short scrambled primers or amplifications based on generic oligoT primer (and its permutations) in conjunction with scrambled primers for messenger ribonucleic acid (“mRNA”) amplifications. In all cases, reaction products are interrogated through post-amplification techniques: arrayed capture probes, electrophoresis, or solution-based deoxyribonucleic acid (“DNA”) specific dyes (e.g., minor groove binders, major grove binders).

Recent advances in attempts to overcome competitive interactions of solution based amplification include separating amplifications in half reactions between solution reactions and interface-based (immobilized) reactions, where half of the primers are in solution, while the other half are immobilized in/on the interface (hydrogels, membranes of organic (nitrocellulose) or inorganic (Al₂0₃) origin). Although advantageous from point of view of separating reactions, there methods are marginally productive, since competition and different efficiencies of amplification still contribute to resulting quantitative biases.

Another approach has been to immobilize both sets of primers on a substrate. The primers are fixed on a substrate so that the primers are still in solution but immobilized in place. The primers are fixed on the substrate via linking compounds. Target DNA is introduced into the solution, denatured, and then allowed to bind with the immobilized primers. Amplification conditions are imposed on the solution to make copies of the target DNA. Denaturing condition are then imposed to separate the target DNA from the extended primers. The initial target DNA is then free to bind with a different set of primers and the process is repeated. Additionally, any primers that have been extended as copies of the target DNA are also able to bind with adjoining primers and also result in additional copies of the target DNA. The process is repeated numerous times until sufficient copies of the target DNA are created from the immobilized primers. Ideally, each cycle of the process results in a doubling of the target DNA that was present before the cycle started. If this ideal is met, then the amplification efficiency is said to equal 100%.

However, the above approach is problematic for target DNA that are 130 base pairs or less in length. DNA that is 130 base pairs or less in length is not very flexible. Instead, the DNA is rather rod-like and resists bending. Therefore, after a primer has been extended to form a copy of the target DNA it is difficult for the extended primer to bend and bind with an adjoining primer. That limits the copies that may be made from the extended primer. This lowers the amplification efficiency for the process and therefore increases the amount of time it takes to amplify the target DNA.

Another problem with the above approach is that often a high density of primers are immobilized on the substrate. This is done to try and increase the probability that when a primer has been extended, there is a complementary primer nearby to which the extended primer can bind. This is done in the hopes that on the next cycle, the nearby complementary primer can also be extended, and, thus, keep the amplification efficiency closer to 100%. A problem with having a high density of primers on the substrate is that if the primers are too close, then the primers tend to sterically hinder the movement of the adjacent primers. Thus, if the primer density is too high, then it is even more difficult for copied target DNA that is 130 base pairs or less in length to bend and bind with an adjoining complementary primer.

Another problem with the above approach is that often the concentration of enzyme, such as a heat stable polymerase, used to extend the primers is high. The enzyme concentration is elevated to increase the probability that if a target DNA does bind to a primer, then there is enzyme available to extend the primer to generate extension products of considerable length (up to several kilo base pairs). Enzyme concentrations up to micromolar concentrations are used. However, the higher the enzyme concentration, then the more likely it is that an error will be introduced into the copy of the target DNA (i.e., the copy will not be an exact duplicate of the complementary target DNA). If all of the copies are exact duplicates, then it is said that the fidelity is 100%. High enzyme concentration can be a problem when looking for the frequency with which mutations occur in a target nucleic acid. If the fidelity is low, then it is difficult to know whether the variation from the target DNA was due to mutation or copying error.

U.S. Pat. No. 5,641,658, filed Aug. 3, 1994, the contents of the entirety of which are incorporated by this reference, discloses a method for performing amplification of a nucleic acid with primers bound to a solid support.

There is a need for methods and devices for amplifying target DNA having 130 base pairs or less.

### BRIEF SUMMARY OF THE INVENTION

Certain embodiments of the invention include a method of building a surface amplification device. The method includes identifying a first primer having an ability to bind to a sense strand of the target nucleic acid. The method also includes identifying a second primer having an ability to bind to an antisense strand of the target nucleic acid. The first primer and the second primer may be connected via a connecting oligonucleotide. The first primer may be attached to a substrate via a first flexible linking compound. The second primer may also be attached to the substrate via a second flexible linking compound. Then, the connecting oligonucleotide may be removed to disconnect the first primer from the second primer. The first primer may be immobilized on the substrate via the first flexible linking compound. The second primer may be immobilized on the substrate via the second flexible linking compound.

Other embodiments of the invention may include a method of amplifying a target nucleic acid. The method may include immobilizing first primers and second primers on a substrate via flexible linking compounds. The flexible linking compounds may be of sufficient length, rotability, and flexibility so that the flexible linking compounds tend to bend and rotate rather than any extension products that may eventually bind to an unextended primer. A sample may be introduced potentially containing the target nucleic acids to the first primers and the second primers. Denaturing conditions may be imposed to separate any target nucleic acids present in the sample into separate target sense strands and target antisense strands. Hybridization conditions may be imposed to anneal any target sense strands and first primers and to anneal any target antisense strands and second primers. Amplification conditions may be imposed to extend any annealed first primers and any annealed second primers. Denaturing conditions may be imposed to separate any target sense strands from any extension products of the first primers and any target antisense strands from any extension products of the second primers. Hybridization conditions may be imposed to anneal any extension products of the first primers with unextended second primers and to anneal any extension products of the second primers with unextended first primers. Amplification conditions may be imposed to extend the unextended second primers and the unextended first primers.

Additional embodiments include a target nucleic acid amplification device. The device may include a substrate. A first primer and a second primer may be attached to the substrate via flexible linking compounds. The flexible linking compounds may be of sufficient length, rotability, and flexibility so that the flexible linking compounds tend to bend and rotate rather than any extension products that may eventually bind to unextended primers.

### BRIEF DESCRIPTION OF THE SEVERAL VIEWS OF THE DRAWINGS

FIG. 1A illustrates one embodiment of a connecting compound connecting a first primer and a second primer during the building of a target nucleic acid surface amplification device.

FIG. 1B illustrates one embodiment of flexible linking compounds attached to a first primer and a second primer.

FIG. 1C illustrates one embodiment of a first primer and a second primer each attached to a substrate via flexible linking compounds.

FIG. 1D illustrates one embodiment of a target nucleic acid amplification device.

FIG. 2A illustrates one embodiment of introducing target nucleic acids to a first primer and a second primer.

FIG. 2B illustrates one embodiment of imposing hybridization conditions to anneal a target sense strand to a first primer.

FIG. 2C illustrates one embodiment of imposing amplification conditions to extend a first primer to form an extension product.

FIG. 2D illustrates one embodiment of imposing denaturing conditions to separate a target sense strand from an extension product.

FIG. 2E illustrates one embodiment of imposing hybridization conditions to anneal an extension product of a first primer to an unextended second primer.

FIG. 2F illustrates one embodiment of imposing amplification conditions to extend an unextended second primer.

FIG. 2G illustrates one embodiment of imposing denaturing conditions to separate the extension products of a first primer from the extension products of a second primer.

FIG. 2H illustrates one embodiment of imposing hybridization conditions to anneal the extension products of extended first and second primers to adjoining unextended first and second primers.

FIG. 2I illustrates one embodiment of imposing amplification conditions to extend unextended adjoining first and second primers.

### DETAILED DESCRIPTION OF THE INVENTION

Embodiments of the invention include methods of building a target nucleic acid surface amplification device as well as the device itself. Embodiments of the invention include methods of amplifying a target nucleic acid.

In one method of building a target nucleic acid surface amplification device, first the target nucleic acid may be identified. Examples of target nucleic acids include DNA or ribonucleic acids (“RNA”). The target nucleic acid may be 130 base pairs in length or less. The target nucleic acid may also be 50 base pairs in length or less. In one embodiment, the target nucleic acid is between 25 and 100 base pairs in length. Generally, the target nucleic acids are double-stranded. Or in other words, each target nucleic acid has a target “sense” strand and a target “antisense” strand. The target antisense strand is complementary and antiparallel to the target sense strand. For example, with the double helix of DNA, one side of the helix is the sense strand and the other side of the helix is the antisense strand. Each target sense strand and target antisense strand has what is referred to as a 5′ end and a 3′ end. The 5′ end of the target sense strand is complementary to the 3′ end of the target antisense strand (assuming the target sense strand is no longer than the target antisense strand). When RNA is the target nucleic acid it may be necessary to first reverse transcribe the RNA to form complementary DNA (“cDNA”) having a target sense strand and a target antisense strand.

Once the target nucleic acid is identified, then primers may be identified that are able to bind to the target nucleic acid. A first primer may be identified that is able to bind to a region of the target sense strand. A second primer may be identified that is able to bind to a region of the target antisense strand. Eventually, the first primer will be extended to form complementary copies of the target sense strand (i.e., duplicate copy of the antisense strand). The first primer and second primer may bind to any region of the respective target sense and antisense strand. However, only the regions downstream from the primers may be copied. Therefore, for example, if the first primer binds to the middle of the target sense strand, then only half of the target sense strand may be copied. It should be understood that the terms “first primer” and “second primer” are not intended to place a greater importance or priority in time to the “first primer,” but rather, the terms are used to differentiate between the two types of primers. Binding may occur by the formation of hydrogen bonds between the nucleotides of the primer and the nucleotides of the sense or antisense strand (i.e., hybridization). First and second primers may be between 15 and 25 nucleotides in length. In one embodiment, the first primers and second primers are 20 to 22 nucleotides in length. However, there is no limitation on the length of the first and second primers.

Identifying the first and second primers may include searching a database, conducting tests on the target nucleic acid, or any method known in the art or later developed for identifying primers. The first and second primers may be prepared using peptide synthesis or by any other method known in the art or later developed.

Once the first and second primers are identified and prepared, then a connecting compound may be identified that has the ability to connect a single first primer to a single second primer. The connecting compound may be able to bind to at least a portion of the first primer while also binding to at least a portion of the second primer.

The length of the connecting compound may be used to set the distance between the first primer and the second primer during the immobilization of the first and second primers. Therefore, it may be desirable to optimize the length of the connecting compound. In one embodiment, the connecting compound may equal the length of the first primer, plus the length of the second primer, plus the length of an additional 1 to 10 nucleotides (i.e., about 3.4 Å to about 34 Å). This may be desirable when the connecting compound binds along the full length of both the first primer and the second primer and when the desired additional length of the extension product is between 1 to 10 nucleotides.

The connecting compound may be a synthetic oligonucleotide. Any method of synthetically creating oligonucleotides known in the art or later developed may be used. In one embodiment, automated oligonucleotide synthesis using phosphoamidite chemistry with a variety of protecting groups (DMT, Fmoc, etc.) Once the connecting compounds are prepared, then they may be connected to the first and second primers to form a primer pair.

Reference will now be made to the figures, wherein like numerals refer to like elements. It should be understood that the drawings are not necessarily to scale.

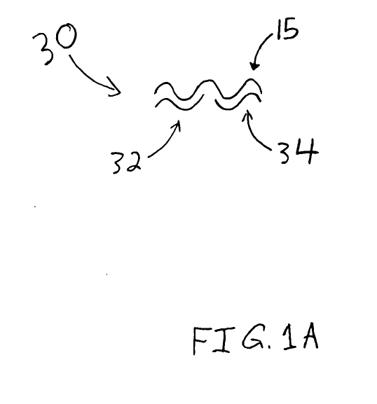

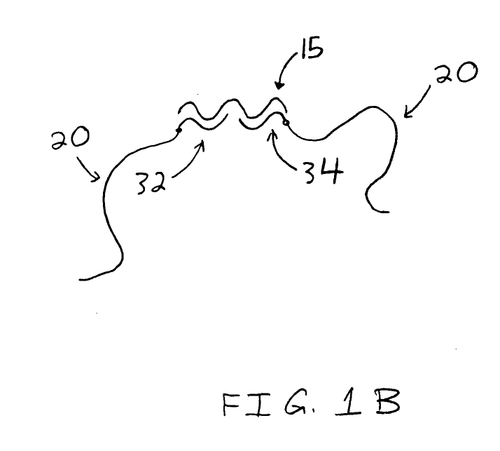

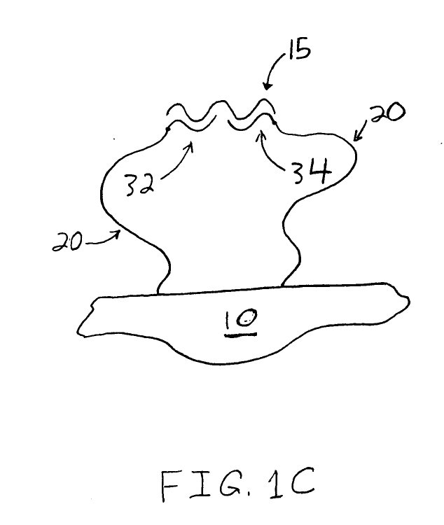

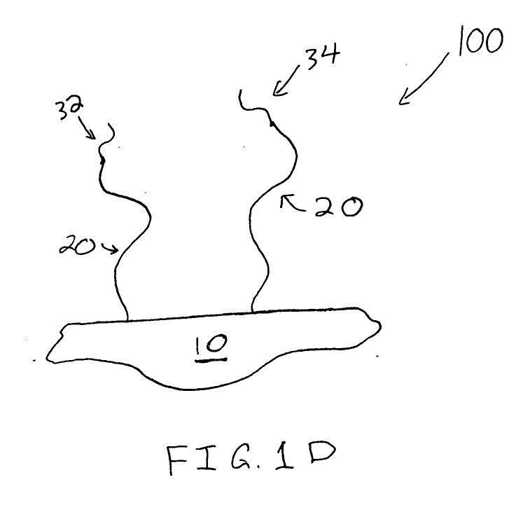

FIG. 1A illustrates one embodiment of a connecting compound 15 connecting a first primer 32 and a second primer 34 to form a primer pair 30. In one embodiment, the connecting compounds 15 may be mixed with the first primers 32 and the second primers 34 in solution to form primer pairs 30.

Either before or after connecting the first primers 32 and the second primers 34 via the connecting compounds 15, the first primers 32 and the second primers 34 may each be attached to a different flexible linking compound 20 as illustrated in FIG. 1B. Flexible linking compounds 20 may be of sufficient length and flexibility so that after the flexible linking compounds 20 are attached to the substrate 10 and after removal of the connecting compound 15, an extension product 52 of the first primer 32 is able to bind to the second primer 34 without bending the extension product 52, or vice versa for an extension product 54 of the second primer 34. In one embodiment, the flexible linking compounds 20 are between about 100 and about 1000 Angstroms in length. The flexible linking compounds 20 may have a length of at least 100 Angstrom (“Å”), at least 200 Å, at least 300 Å, at least 400 Å, at least 500 Å, at least 600 Å, at least 700 Å, at least 800 Å, at least 900 Å, or at least 1000 Å.

Flexible linking compounds 20 may be made from any material with sufficient flexibility and rotability. For examples, flexible linking compounds 20 may include polysaccharides, or organic linear polymers, such as polyethylene glycol (“PEG”), polyacrylamide, and uncross-linked derivatives. Flexible linking compounds 20 may be functionalized in order to achieve immobilization on the surface of substrate 10. Flexible linking compounds 20 may have features on the non-immobilized end for attachment to first primers 32 and second primers 34. Attachment between flexible linking compounds 20, first primers 32 or second primers 34, and substrate 10 may be accomplished by chemical or affinity attachment or by any other method known in the art or later developed.

Next, as illustrated in FIG. 1C, flexible linking compounds 20 may be attached to substrate 10. Connecting compounds 15 allow for optimal placement of primer pairs 30 via flexible linking compounds 20. Due to flexibility and rotability, flexible linking compounds 20 may tend to attach to substrate 10 at locations that reduce steric hindrances and that avoid straining the primer pairs 30 or the flexible linking compounds 20.

It should be understood that numerous primer pairs 30 may be seeded onto substrate 10 via flexible linking compounds 20. The concentration of primer pairs 30 in solution may be controlled to determine the average density of primer pairs 30 on the surface of substrate 10. The correlation between primer pair 30 concentration in solution and the resulting average density of primer pairs 30 on the surface of substrate 10 may be determined experimentally. For example, a solution with a known concentration of labeled primer pairs 30 may be formed over substrate 10. The primer pairs 30 that attach to the surface of substrate 10 may then be detected and quantified. A correlation could then be established between primer pair 30 concentration in solution and average density on the surface of substrate 10. The average distance between primer pairs 30 could then be determined.

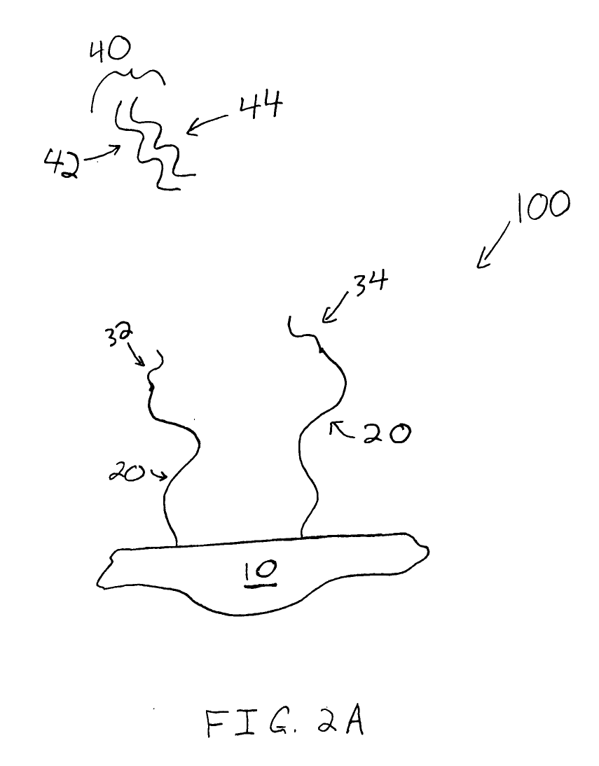

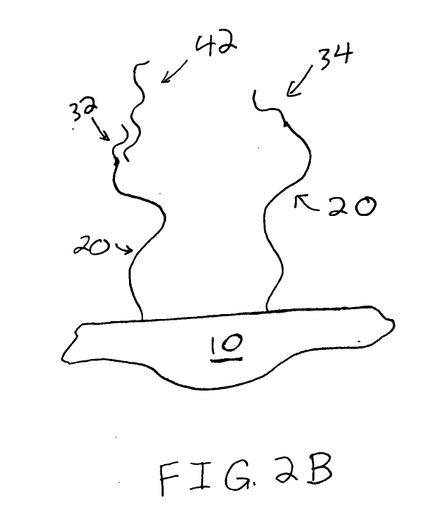

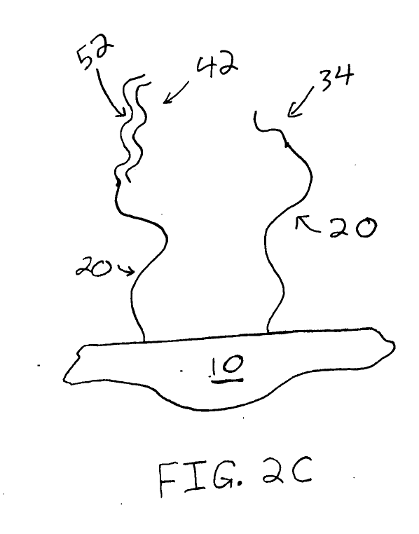

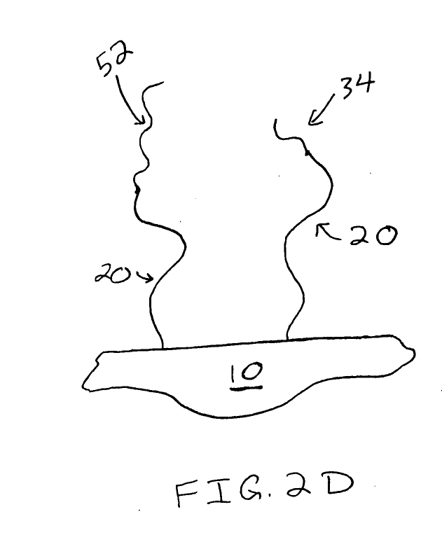

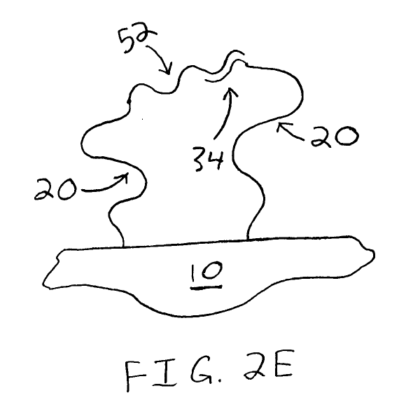

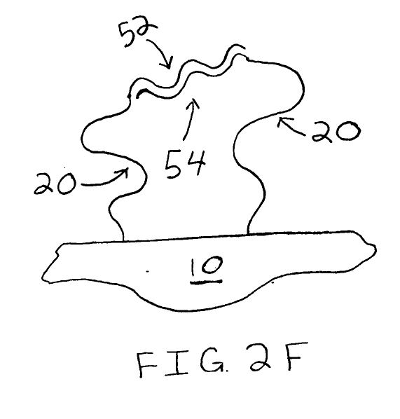

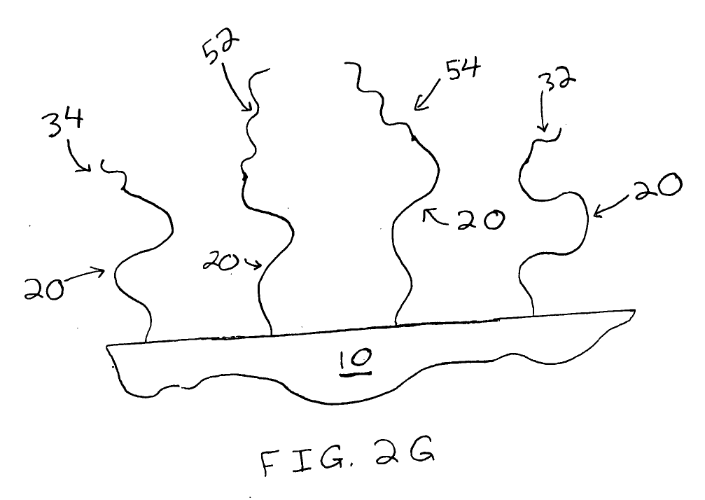

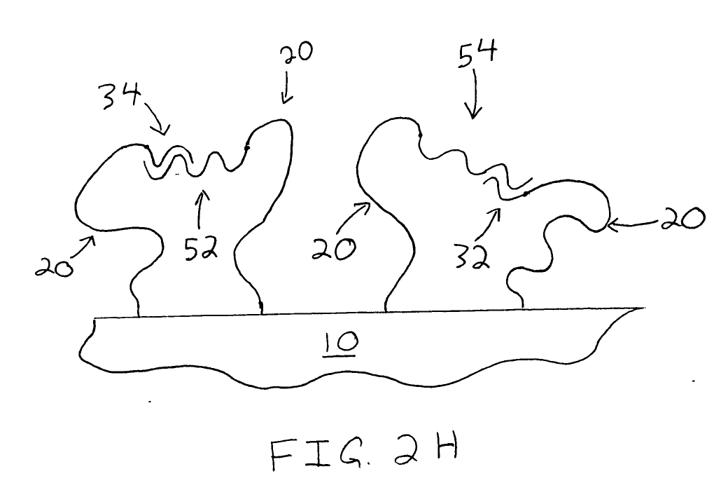

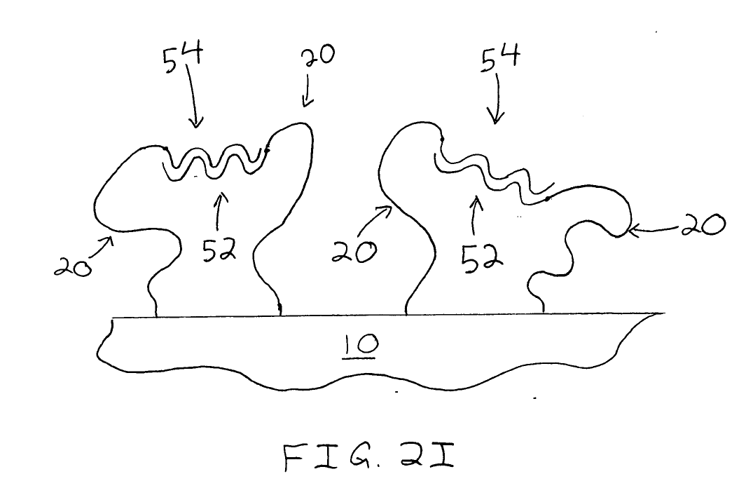

The density of primer pairs 30 on the surface of substrate 10 may be controlled to provide optimal distance between primer pairs 30. For example, adjacent primer pairs 30 may be placed close enough together so that an extension product 52 of an extended primer 32 (see FIG. 2H) is able to bind to an unextended primer 34 from an adjoining primer pair 30, without inducing strain into any of the oligonucleotides. Thus, because strain may be avoided or reduced, the amplification efficiency of each amplification cycle may be improved. At the same time, the adjoining primer pairs 30 may be far enough apart that one primer from one primer pair 30 does not sterically hinder the ability of another primer from a different primer pair 30 to bind with a target nucleic acid or the extension product of a primer. In one embodiment, the average density of primer pairs 30 is 10¹⁰ per square centimeter of the surface of substrate 10. In one embodiment, the distance between primer pairs 30 is approximately the sum of the rotational radii of first primers 32 and second primers 34. That distance may be reduced up to 20 Å. In another embodiment, the average distance between the primer pairs 30 is set equal to the average distance between the first primer 32 and the second primer 34 of any given primer pair 30. Thus, the first primer 32 of a primer pair 30 would be just as likely to interact with the second primer 34 of an adjoining primer pair 30 as with the initially paired second primer 34. Experiments may be conducted to determine the average distance between primer pairs 30 that result in the highest amplification efficiency.

Regarding substrate 10, substrate 10 may be any substrate known in the art for immobilizing primers. For example, substrate 10 may be a bead, such as a latex bead, or a a flat surface, such as a glass or polymer surface. The substrate 10 may be made from a material that is compatible with, or may be made to be compatible with, flexible linking compounds 20. Substrate 10 may also be designed to enhance the detection of target nucleic acid amplification. Possible enhanced interfaces include: membranes, thin film planar waveguides, fiber optics guides, surface modifications with polymeric or inorganic porous beads, nanoparticles and nanocavities, and efficient selective excitation substrates (e.g., evanescent field).

After the flexible linking compounds 20 are attached to substrate 10, then connecting compounds 15 may be removed, as illustrated in FIG. 1D. Connecting compounds 15 may be removed by unhybridizing connecting compounds 15 from first primers 32 and second primers 34. The surface amplification device 100 may then be used for target nucleic acid amplification. For example, surface amplification device 100 may be used for detecting target nucleic acids in a sample.

Surface amplification device 100 may also be used for detecting a frequency of mutations in a target nucleic acid, such as a single nucleotide polymorphism (“SNP”). For example, either first primer 32 and/or second primer 34 may be designed to be complementary to a potential mutation in the target nucleic acid. If the mutation is present in a sample containing the target nucleic acid, then the mutated target nucleic acid will be amplified. If the mutation is not present, then the target nucleic acid will not be amplified. Real-time detection and quantitative analysis may be used to determine the frequency at which the mutation occurs in the target nucleic acid.

It should be understood that surface amplification device 100 may be part of a larger device and/or system. For example, surface amplification device 100 may be part of a flow system where the surface of substrate 10 is periodically or continuously flushed. Surface amplification device 100 may be part of an immersion system where substrate 10 is immersed in a bath containing any necessary reagents in solution.

Surface amplification device 100 may be part of a microarray. In this embodiment, the surface of the microarray may be the surface of substrate 10. Spots may be formed on the surface of the microarray having first primers 32 and second primers 34 immobilized via flexible linking compounds 20. The necessary reagents and samples may be administered drop-wise to the individual spots. In one embodiment, each spot of the microarray may designed to amplify a different target nucleic acid. In another embodiment, all of the spots may be designed to amplify the same target nucleic acid.

Surface amplification device 100 may be incorporated into a self-contained reaction cartridge. The reaction cartridge may contains all of the necessary reagents needed to perform an assay. The reaction cartridge may have a port for introduction of the sample and separate isolated chambers for buffers, enzymes, and detection agents (e.g., dyes or labeled oligonudeotides). At programmed intervals, reagents may be released from the reagent chambers and delivered to a central reaction site, containing the sample (and possibly the target nucleic acids) and first primers 32 and second primers 34 immobilized on substrate 10 via flexible linking compounds 20.

In an alternative embodiment of building surface amplification device 100, flexible linking compounds 20 may be attached to the surface of substrate 10 prior to attaching primer pairs 30 to the flexible linking compounds 20. In this embodiment, the optimal density of flexible linking compounds 20 needed to mesh with the primer pairs 30 may be calculated. After the primer pairs 30 are attached to the flexible linking compounds 20, then the connecting compounds 15 may be removed.

In another alternative embodiment of building surface amplification device 100, connecting compounds 15 may not be used. Instead, flexible linking compounds 20 may be pre-seeded on the surface of substrate 10 with a desired density. First primers 32 and second primers 34 may be introduced into solution in equimolar quantities, without being paired together, and attached to the flexible linking compounds 20.

Turning now to other embodiments of the invention, embodiments of the invention include methods of amplifying a target nucleic acid. The methods of amplifying a target nucleic acid may also be used for detecting whether a target nucleic acid is present in a sample.

In one method of amplifying a target nucleic acid, first primers and second primers may be immobilized on a substrate via flexible linking compounds. The flexible linking compounds may be of sufficient length, rotability, and flexibility so that the flexible linking compounds tend to bend and rotate rather than any extension product of one primer that may eventually bind to another primer. The flexible linking compounds and the primers attached thereto may be optimally seeded to reduce steric hindrances between the first primers and second primers. They may also be optimally seeded to promote annealing between extension products of first or second primers and adjoining unextended first or second primers. The first primers and second primers may be immobilized according the embodiments heretofore discussed and/or illustrated in FIGS. 1A-1D.

Next, a sample potentially containing target nucleic acids may be introduced to the immobilized first primers and second primers. FIG. 2A illustrates target nucleic acids 40 introduced in solution to surface amplification device 100. Target nucleic acids 40 include target sense strands 42 and target antisense strands 42. Target nucleic acids 40 may be 130 base pairs in length or less. Target nucleic acids may be 50 base pairs in length or less. Target nucleic acids 40 may be between 25 and 100 base pairs in length.

After the sample is introduced to surface amplification device 100, then denaturing conditions may be imposed to separate any target nucleic acids 40 present in the sample into separate target sense strands 42 and target antisense strands 44. Denaturing conditions may be imposed by elevating the temperature, changing the ionic strength, and/or altering the pH of the solution. Any method known in the art, or later developed, for denaturing nucleic acids may be used.

After any target nucleic acids 40 are denatured, then hybridization conditions may be imposed to anneal any target sense strands 42 and first primers 32 and to anneal any target antisense strands 44 and second primers 34. FIG. 2B illustrates a target sense strand 42 annealed to a first primer 32. It should be understood, that although target antisense strand 44 is not depicted in FIG. 2B, target antisense strand 44 could also be annealed to the illustrated second primer 32 or to a non-illustrated second primer 32. Hybridization conditions may be imposed by lowering the temperature, changing the ionic strength, and/or altering the pH of the solution. Any method known in the art, or later developed, for hybridizing nucleic acids may be used.

Next, amplification conditions may be imposed to extend any annealed first primers and any annealed second primers. FIG. 2C illustrates that primer 32 may be extended to form extension product 52. Extension product 52 is complementary to target sense strand 42 and, unless there has been an error, identical to at least a portion of target antisense strand 44. Amplification conditions may be imposed by adding any suitable reagents for amplification that may be necessary (e.g., thermal stable polymerase, nucleotides, reagents, and buffers). Any method known in the art for imposing amplification conditions may be used.

In one embodiment, the enzyme (e.g., thermal stable polymerase) that is used to extend first primers 32 and second primers 34 has a concentration that is lower than the concentration of target nucleic acids 40 present in the sample. The enzyme concentration may be low because embodiments of the present invention may have an increased amplification efficiency compared to surface amplifications without the benefit of embodiments of the present invention. Additionally, the shorter extension products 52 and 54 are, then the less enzyme needed. Thus, when extension products 52 and 54 are 130 base pairs or less, then the amount of enzyme needed may be reduced. The lower enzyme concentration may increase the fidelity of the amplification. In another embodiment, the concentration of enzyme used may be an order of magnitude less than the amount of enzyme commonly used with surface amplifications with immobilized primers. The increased fidelity of certain embodiments of the invention may make surface amplification device 100 useful in identifying mutations in target nucleic acids that have a low frequency of mutation.

After amplification, denaturing conditions may be imposed to separate any target sense strands 42 from any extension products 52 of first primers 32 and any target antisense strands 44 from any extension products 54 of second primers 34, such as illustrated in FIG. 2D. The same methods used initially to impose denaturing conditions on target nucleic acids 40 may be used again.

Hybridization conditions may then be imposed, such as illustrated in FIG. 2E, to anneal any extension products 52 of first primers 32 with unextended second primers 34 and to anneal any extension products 54 of second primers 34 with unextended first primers 32. Flexible linking compounds 20 may result in substantially avoiding bending any extension products 52 or 54 during hybridization to either second primers 34 or first primers 32, respectively. The same methods used to initially impose hybridization conditions may be used again. Although not illustrated in FIG. 2E, it should be understood that while any extension products 52 and any extension products 54 are annealing to unextended primers, the initial target sense strand 42 may also be annealing to extended or unextended first primers 32. The same is true for the initial target antisense strand 44 and second primers 34.

After hybridization, amplification conditions may be imposed to extend unextended second primer 34, such as illustrated in FIG. 2F, to form extension product 54. The same methods used initially to impose amplification conditions in order to extend first primer 32 may be used again.

The process of imposing denaturing conditions, imposing hybridization conditions, and imposing amplification conditions may be repeated a number of times until substantially all of the first primers 32 and the second primers 34 have been extended. FIGS. 2G-2I illustrate how extension products 52 and 54 may result in additional extension products 52 and 54 after an additional cycle of denaturing, hybridizing, and amplifying. The shorter extension products 52 and 54 are, the less time that is needed for denaturing, hybridizing, and amplifying. When extension products 52 and 54 are 130 base pairs or less, the time required for amplifying target nucleic acids 40 may be relatively short.

Methods of assaying target nucleic acids 40 may include monitoring substrate 10 to detect extension products 52 and 54. Detecting extension products 52 and 54 may indicate the presence of target nucleic acids 40 in the sample. A lack of detecting extension products 52 and 54 may indicate the absence of target nucleic acids 40 in the sample. A variety of detection systems may be used.

For example, fluorescence-based systems may be used. In one embodiment, fluorescence resonance energy transfer (“FRET”) may be used. In one possible use of FRET, the 3′ ends of each of the first primers 32 and second primers 34 are labeled with a fluorescent moiety. The first primers 32 may be labeled with a donor moiety and the second primers 34 labeled with an acceptor moiety, or vice versa. The donor moiety may be a fluorophore and the acceptor moiety may quench the frequency of light emitted by the donor moiety. In this embodiment, when an extension product 52 of a primer 32 binds to a primer 34, then the donor moiety may be sufficiently close to the acceptor moiety that, upon excitation of the donor moiety, FRET occurs and the intensity of emitted light is reduced. Additionally, U.S. Publication 2002/0197611, published Dec. 26, 2002, the contents of the entirety of which are incorporated by this reference, discloses methods of labeling primers. Thus, it is possible to detect when either a first primer 32 or second primer 34 has been extended. Thus, it is possible to detect whether target nucleic acids 40 are present in the sample.

Additionally, multiple target nucleic acids 40 may be amplified and detected. For example, a first set of first primers 32 and second primers 34 may be complementary to a first target nucleic acid. A second set of first primers 32 and second primers 34 may be complementary to a second target nucleic acid. The first set of primers may be labeled in a manner that is detectably distinguishable from the second set of primers (e.g., donor fluorophores that fluoresce at different wavelengths).

Other fluorescence-based systems may also be used. For example, the intercalating dyes would detectably fluoresce, upon excitation, if double-stranded nucleic acids were present in surface amplification device 100. However, intercalating dyes are generally non-specific. Therefore, it would be unclear based just on the fluorescence alone, whether it was extension product 52 and 54 binding together or some other nucleic acids.

Additionally, non-fluorescence-based systems may also be used for monitoring substrate 10. For example, if target nucleic acids 40 are present, then the amount of target nucleic acids 40 may be sufficiently increased to actually be a detectable amount of mass for use with tools such as mass spectrometers.

In addition to qualitative analysis, methods of assaying target nucleic acids 40 may include performing real-time quantitative analysis of target nucleic acids 40 based upon data collected during monitoring substrate 10.

As discussed above, embodiments of the present invention may have increased amplification efficiency, fidelity and reduced amplification time. Thus, embodiments of the present invention may make surface amplification much more feasible as a method of amplifying target nucleic acids.

The aforementioned methods and devices are not meant to be limiting. Other steps known in the art or developed in the future for amplifying specific types of target nucleic acids may also be added to the above methods and/or implemented with the above devices.

Specific embodiments have been shown by way of example in the drawings and have been described in detail herein. However, it should be understood that the invention is not intended to be limited to the particular forms disclosed. Rather, the invention is to cover all modifications, equivalents, additions, and alternatives falling within the spirit and scope of the invention as defined by the following appended claims.

What is claimed is:

1. A method of building a surface amplification device, said method comprising:
identifying a first primer having an ability to bind to a sense strand of a target nucleic acid;
identifying a second primer having an ability to bind to an antisense strand of said target nucleic acid;
connecting said first primer and said second primer via a connecting oligonucleotide;
attaching said first primer to a substrate via a first flexible linking compound;
attaching said second primer to the substrate via a second flexible linking compound;
removing said connecting oligonucleotide to disconnect said first primer from said second primer and thereby leaving said first primer immobilized on said substrate via said first flexible linking compound and thereby leaving said second primer immobilized on said substrate via said second flexible linking compound.

2. The method according to claim 1, further comprising identifying a connecting oligonucleotide having an ability to connect said first primer to said second primer.

3. The method according to claim 2, further comprising identifying a connecting oligonucleotide able to bind to at least a portion of the first primer while also binding to at least a portion of the second primer.

4. The method according to claim 1, further comprising creating a synthetic oligonucleotide having the ability to connect the first primer to the second primer.

5. The method according to claim 1, wherein connecting said first primer and said second primer via said connecting oligonucleotide comprises selecting a connecting oligonucleotide of sufficient length to optimize the immobilized distance between said first primer and said second primer.

6. The method according to claim 5, wherein selecting a connecting oligonucleotide of sufficient length comprises selecting a connecting oligonucleotide having a length equal to the length of said first primer, plus the length of said second primer, plus the length of an additional 1 to 10 nucleotides.

7. The method according to claim 1, further comprising selecting a first flexible linking compound and a second flexible linking compound of sufficient lengths so that, after said removal of said connecting oligonucleotide, an extension product of said first primer is able to bind to said second primer without bending said extension product of said first primer.

8. The method according to claim 1, wherein attaching said first primer to said substrate via said first flexible linking compound comprises attaching said first primer to said first flexible linking compound and then attaching said first flexible linking compound to said substrate.

9. The method according to claim 1, further comprising identifying a target nucleic acid to be amplified with the surface amplification device.

10. A method of amplifying a target nucleic acid, said method comprising:
immobilizing first primers and second primers on a substrate via flexible linking compounds, wherein said flexible linking compounds are of sufficient length, rotability, and flexibility so that said flexible linking compounds tend to bend and rotate rather than any extension product that may eventually bind to an unextended primer;
introducing a sample potentially containing said target nucleic acid to said first primers and said second primers;
imposing denaturing conditions to separate any target nucleic acids present in said sample into separate target sense strands and target antisense strands;
imposing hybridization conditions to anneal any said target sense strands and said first primers and to anneal any said target antisense strands and said second primers;
imposing amplification conditions to extend any annealed first primers and any annealed second primers;
imposing denaturing conditions to separate any said target sense strands from any extension products of first primers and any target antisense strands from any extension products of second primers;
imposing hybridization conditions to anneal any said extension products of first primers with unextended second primers and to anneal any said extension products of second primers with unextended first primers; and
imposing amplification conditions to extend said unextended second primers and said unextended first primers.

11. The method according to claim 10, further comprising repeatedly imposing denaturing conditions, imposing hybridization conditions, and imposing amplification conditions until substantially all of said first primers and said second primers have been extended.

12. The method according to claim 10, wherein immobilizing said first primers and said second primers on said substrate via said flexible linking compounds comprises optimally seeding said first primers and said second primers to reduce steric hindrances between said first primers and said second primers and to promote annealing between extension products of first or second primers and adjoining unextended first or second primers.

13. The method according to claim 10, wherein annealing any said extension products of first primers with said second primers and annealing any said extension products of second primers with said first primers comprises substantially avoiding bending any said extension products of first primers and any said extension products of second primers.

14. The method according to claim 10, wherein separating any target nucleic acids present in said sample into separate target sense strands and target antisense strands comprises separating any target nucleic acids present in said sample into separate target sense strands of 130 base pair or less, and target antisense strands of 130 base pair or less.

15. The method according to claim 10, wherein imposing amplification conditions to extend any annealed first primers and any annealed second primers comprises using an enzyme concentration that is lower than the concentration of target nucleic acids potentially present in said sample.

16. The method according to claim 10, further comprising monitoring said substrate to detect extension products of any of said first primers and said second primers, where detecting extension products indicates the presence of said target nucleic acids in said sample and a lack of detecting extension products indicates the absence of said target nucleic acids in said sample.

17. The method according to claim 16, further comprising performing real-time quantitative analysis of said target nucleic acid based upon data collected during monitoring said substrate.

18. A target nucleic acid surface amplification device comprising:
a substrate; and
a first primer and a second primer attached to the substrate via flexible linking compounds, wherein said flexible linking compounds are of sufficient length, rotability, and flexibility so that said flexible linking compounds tend to bend and rotate rather than any extension products that may eventually bind to unextended primers.

19. The surface amplification device of claim 18, wherein said flexible linking compounds have a length selected from the group consisting of: at least 100 Angstrom (“Å”), at least 200 Å, at least 300 Å, at least 400 Å, at least 500 Å, at least 600 Å, at least 700 Å, at least 800 Å, at least 900 Å, and at least 1000 Å.

20. The surface amplification device of claim 18, further comprising multiple pairs of first primers and second primers, wherein the average distance between said primer pairs equals about the length of two flexible linking compounds, plus the length of a first primer, plus the length of a second primer, plus about 30 Å.
